(19) United States
(12) Patent Application Publication
(10) Pub. No.: US 2015/0328275 A1
(43) Pub. Date: Nov. 19, 2015

(54) DIETARY SUPPLEMENT FOR PET MAMMALS

(71) Applicant: GRATUK TECHNOLOGIES PTY LTD, New South Wales (AU)

(72) Inventor: Malcolm BALL, New South Wales (AU)

(21) Appl. No.: 14/382,955

(22) PCT Filed: Mar. 5, 2013
(86) PCT No.: PCT/AU2013/000200

§ 371 Date: Sep. 4, 2014

(30) Foreign Application Priority Data
Mar. 5, 2012 (AU) 2012900845

(51) Int. Cl. A61K 36/899; A61K 9/14; A23K 1/00; A23K 1/18; A23K 1/16

(52) U.S. Cl. CPC A61K 36/899; A23K 1/1853; A23K 1/1643; A23K 1/002; A61K 9/14

(57) ABSTRACT

Use of dietary fibre material extracted from sugar cane in the manufacture of a food product that is formulated to ameliorate the effects of diabetes in pet animals.

### TECHNICAL FIELD

The invention relates to the field of pet food supplement manufacture. In particular, the invention relates to a dietary supplement, the use of said supplement in the diet of a dog, and the method of manufacture of said supplement.

### BACKGROUND OF THE INVENTION

As is the case for humans, diabetic conditions are becoming a more common health problem in pets who act as companion animals for humans, including canines and felines.

There are three types of diabetes: mellitus, insipidus and gestational. In canines, diabetes mellitus is the most common, followed by gestational diabetes, whilst cats only appear to suffer from diabetes mellitus. Both of these conditions result in the animal being unable to regulate blood glucose levels unassisted.

In the case of gestational diabetes a pregnant bitch loses the ability to regulate blood glucose due to hormone imbalances. Normal regulation is usually regained after the birth of the puppies, however the condition needs to be managed while the bitch is pregnant, as the condition can be fatal. In addition, gestational diabetes results in a number of negative effects on the health of the puppies.

Diabetes mellitus is a chronic condition, and in canines it is homologous to Type-1 diabetes in humans, whilst in felines it is most often homologous to Type-2 diabetes. The causes of diabetes in animals is uncertain, though the most common causes of diabetes mellitus in dogs and cats cited in the literature are obesity and hyperlipemia, caused by high fat diets and poor gut health, resulting in autoimmune conditions. These conditions have also been reported as major contributors to the development of diabetic conditions in humans, suggesting common environmental causes for the three species.

In many studies on diabetic conditions, canines have been used as models for humans. While there are a number of oral drugs available to humans, treatment of diabetes in dogs and cats is usually restricted to diet control and sub-cutaneous injection of human insulin, which does not necessarily result in good daily blood glucose levels.

In recent years the incidence of diabetes mellitus in dogs has dramatically increased (from 19 per 10,000 individuals to 50 per 10,000 individuals) and it is estimated that as many as one third of all dogs may be at risk. Treatment of the disease is complicated and expensive so any method of reducing risk and/or alleviating symptoms is of significant benefit to dog owners.

The incidence of diabetes mellitus in felines has been reported to be as high as 74 per 10,000 individuals, possibly higher as a cat is usually not diagnosed with diabetes until severe symptoms occur. As the majority of cats have type 2 diabetes treatments that help regulate blood glucose levels and increase insulin sensitivity are especially useful. Additionally many of the animals can be treated by diet control alone.

Commercial pet foods are typically not formulated with the specific treatment of diabetic conditions in mind, so these diets alone are not likely to reduce the animal's risk of developing diabetes mellitus. In particular, it is thought that the fibre sources used in many commercial pet foods are unsuitable to mitigate diabetic risk. There is also a growing market pull for more “natural” pet food products.

Canine physiology tends to mimic human physiology in many ways. Canine versions of common food allergies and intolerances have been reported with respect to wheat, dairy, soy, and oats, amongst others. It is thought that food allergies represent 10% of all allergies in dogs and it is estimated that around 20% of all dogs suffer from allergies of some kind. Feline physiology is not as closely linked to humans as they are more dedicated carnivores than omnivores like humans and canines, however cats also suffer food allergies though not to the same extent as dogs. Even so, wheat is a common allergen amongst felines.

The proportion of pets suffering food allergies is therefore significant, and coupled with the fact that poor gut health is a potential cause of diabetes in animals, a hypoallergenic treatment would be preferred. Currently all diet related treatments for pets involve high fibre foods, however this fibre is most often derived from wheat and/or oats. It is also known that not all high fibre diets work to control blood glucose level.

Accordingly, it is an object of the invention to improve the delivery of dietary fibre to the diet of canines and felines that are at risk of diabetic conditions, that ameliorates the identified disadvantages of the prior art.

### SUMMARY OF THE INVENTION

According to one aspect of the invention, there is provided the use of dietary fibre material extracted from sugarcane in the manufacture of a companion animal food product that is formulated to ameliorate the effects of diabetic conditions.

Preferably, the sugarcane fibre is prepared via a process including the steps of: subjecting the sugar cane material to at least one wet diffusion step to separate sugars from a residual fibre material; and subjecting the residual fibre material to a rapid, low-heat or short exposure time drying process thereby to enhance the water retention properties of said residual fibre product.

There are a number of advantages to using dietary fibre material extracted from sugar cane in the way described above. Firstly, no adverse allergic effects in companion animals have ever been recorded with this source. Also, this fibre source has been shown to improve gut lining health over and above other sources of fibre. It contains benefits of both soluble and insoluble fibre and has a ratio of fibres that more accurately represents natural foods than other products. It is also high in other micronutrients such as iron and has the ability to protect antioxidants.

Fibres separated from grasses such as sugarcane have several advantageous properties compared to incomplete (not ‘whole plant’) fibres such as wheat bran or cellulose pulp—the two most common sources of fibre in pet foods. The fibre from sugarcane is a true lignose, hemicellulose and cellulose combination similar to the total dietary fibres found in vegetables. Additionally, even sugarcane fibre is classed as almost entirely insoluble fibre, it has many of the properties of soluble fibre as well it has a high water binding capacity (up to 8-10 times by weight) and a probiotic effect. Insoluble fibres traditionally are known to have little or no effect on blood glucose levels it has been observed that when prepared correctly sugarcane fibre can have profound benefits on postprandial blood glucose levels. This is most likely a combination of the fact that the hemicellulose fraction of the fibre has soluble components that are released during digestion and that when prepared correctly the fibre retains a number of biologically active molecules.

In addition, when this fibre source is prepared via the process as described herein, the fibre tends to retain its functionality with respect to canine and feline diabetes to a greater level due to the retention of biologically active molecules in the fibre. The fibre source also provides the correct dietary fibre to address this deficiency in the majority of the canine and feline population.

The invention also allows more flexible product formats to be developed, in particular ones that allow the owners of animals suffering diabetic conditions to address to deficiency in their own way, especially when provided with the correct type of fibre in a relatively easy-to-use format. Owners no longer have to rely on food manufacturers to generate high fibre foods that their pets will find palatable.

Preferably, the wet extraction step is a diffusion extraction, done under relatively low-shear conditions. The optimal wet extraction step temperature is in the range 25° C. to 70° C.

According to another aspect of the invention, there is provided a food product formulated to ameliorate the effects of companion animal diabetes; said food product containing dietary fibre material extracted from sugar cane, said dietary fibre material preferably having been prepared according to the steps defined above.

According to another aspect of the invention, there is provided a method of treatment of the effects of canine diabetes in a companion animal by feeding to said companion animal a food product incorporating dietary fibre material extracted from sugar cane; said dietary fibre material preferably having been prepared according to the method defined above.

Now will be described, by way of a particular, non-limiting example, a preferred embodiment of the invention.

### DETAILED DESCRIPTION OF THE INVENTION

The current invention takes advantage of the properties of a dietary fibre isolate produced from sugar cane, in such a way that minimal destruction of the bioactive molecules occurs.

The method of preparation of the fibre material from sugar cane is broadly similar to that described in WIPO patent document no. WO2011/035381 by KFSU Pty Ltd, which is incorporated herein by reference. However, the process according to the present invention may be defined as having the following essential features:
1. A sugar cane size reduction step;
2. A relatively ‘gentle’ aqueous extraction stage that separates the fibre from other sugar cane fractions, including the sugar fraction, without causing degradation of the fibre functionality or significant loss of bioactivity; and
3. A relatively gentle drying step that minimises degradation of the fibre functionality and maximises bioactivity of the retained micronutrients.

It is preferred that the extraction step be an aqueous diffusion extraction performed at a relatively neutral pH. It is also preferred that the drying step be a rapid vortex drying operation that, as may be achieved via a low temperature, vortex dryer, such as that supplied by Tensei in Japan (www.tensei-j.com).

It is understood that adequate dietary fibre is important to the healthy function of the canine and feline digestive system. It is also known that dietary fibre levels can have an influence on the likelihood of development of diabetic conditions in humans. It is also thought that most commercial canine diets are deficient in dietary fibre, use fibre sources that have been shown to be ineffective and/or use fibre sources known to cause allergies and intolerances in canines, such as wheat and oats.

The invention provides for the use of sugarcane fibre in the formulation of canine foods or diets that seek to reduce the risk of development of diabetic conditions. When prepared according to the invention, this fibre source, and the foods incorporating it, has a number of advantages over other fibre sources and food, including that:
It is relatively hypoallergenic;
It contains both insoluble and soluble fibre properties in beneficial proportions for dietary intake;
It contains a number of bioactive molecules that beneficially affect blood glucose levels and intestinal health to a greater degree than other fibre sources;
It can be prepared in a ‘chemical-free’ manner and contain no harmful trace elements, unlike fibre from other sources such as chemically modified starch; and
It can be prepared in such a way as to retain the micronutrients and active molecules found in the “molasses” component of sugarcane;

It is also known that too much fibre in the diet of dogs can have several negative side effects including but not limited to constipation, diarrhoea, and bad flatulence. In one embodiment, where the fibre product is added as a supplement to the animal's diet, the animal's dietary fibre intake can be more easily controlled by the owner, without requiring the owner to otherwise change the animal's diet.

The supplement is also classed as a natural food which is increasingly important to many owners.

The embodiments of the invention can take a number of forms, each with several advantages for users.

In this document:
The term “carrier” is used to describe a palatable ingredient that can be combined with the sugar cane fibre to make consumption of the fibre easier for the individual. The carrier may contain protein or other nutrients, including, but not limited to, juices, puddings, sauces, and yogurts.
The term “inert filler” refers to any product used to add bulk to the sugar cane fibre to allow for ease of packaging or consumption. The inert filler may contain flavours or nutrient, and may contain other sources of natural fibre for the purpose of improving mouth feel or soluble/insoluble fibre ratios.
The term “pellet” refers to any dry form of the fibre, including, but not limited to: a dried pill; a “grain”-style sprinkle; a compacted powder that may be added to the medium of the user's choice; an extruded kibble that may be used as a treat or as a addition to other foods; or a textured “jerky”-style treat like those commonly available as treats for dogs.
The term “flavouring” refers to a palatable ingredient, in solid or liquid form, incorporated in the canine food to induce a desire for the animal to consume the food, and which may contain protein or other nutrients; for example, animal liver extracts, digests, broths, purees or other offals.

All of the examples below can optionally be formulated with additional vitamins and bioactive molecules, such as stevia. Preferably any added nutrients would be sourced from natural ingredient to that a “natural” descriptor may be maintained for the final product.

### Example 1

In this example, 0.5 g of the active fibre is added to a flavouring medium and/or carrier, and then pressed into a pellet. Each pellet provides 50% of the animal's daily fibre requirements, as well as helping to manage the blood glucose levels (per 5 kg of bodyweight) per meal. The owner is instructed to dose the animal accordingly. If the animal is on a fibre diet of another kind then this pellet can be used in conjunction with other food thereby reducing the risk of having too much fibre in the diet of the animal. However if there is no other fibre provided to the animal then the dose may be doubled without negative effects. The pellet is provided to the animal by either mixing it in other food, or may be given as a treat immediately before or after being fed a main meal.

### Example 2

In this example the active fibre is mixed with a flavour and an inert liquid selected to achieve the viscosity of a paste (approximately 0.5 g per 3 ml of liquid). The paste is dispensed from a graduated cylinder that is airtight and includes a convenient dispensing mechanism (for example a sterile syringe). The paste may be mixed into the animal's food or directly placed into the mouth by the owner immediately before or after a meal. This method of delivery has the added benefit that it may be used to coat fresh food meals such as whole meat or offal, or may be added to table scraps by the owner.

### Example 3

In this example the active fibre is mixed with a dry flavour component and an inert filler to form easy-to-use granules. The mixture is formulated such that the fibre dose (0.5 g) would be equal to one scoop of the granules, and the appropriate number of scoops would be applied to wet food based on the weight of the animal. This method of delivery is best suited to a weight loss diet as the granules can be mixed with water to act as a filler (thereby allowing less of the food to be used each meal). This example is also more useful for dogs that weigh less than 5 kg as a partial dose may be easily measured by the owner.

### Example 4

In this example the fibre material is supplied as an ingredient for other manufacturers of high-fibre foods for the gluten-free or ‘digestive eating’ markets. This example provides several benefits for potential food manufacturers/suppliers:
If the fibre material is used to replace wheat or oat fibres then the product may be labelled as hypo-allergenic.
The fibre material supports the use of “all natural” marketing claims for the foods.
The fibre material provides other health benefits compared with other fibre sources, allowing the food manufacturer to potentially make more substantive claims.

The water retention capacity of the sugar cane, prepared as described above, is far greater than most commercial fibre sources. By using the active fibre in foods the manufacturer can reduce calorific content per kg of food. This may also result in a significant commercial saving for the manufacturer.

Pet food products and methods according to the invention make use of the unique qualities of cane based crops, particularly whole sugarcane, that have been prepared using a chemical-free, low-heat procedure. This makes it easy and convenient to use while still retaining the beneficial nutrients and bioactive molecules in the food.

The products and methods address several problems associated with poor fibre consumption in pet animals, as well as having a positive impact on diabetic conditions, while also contributing to the elimination of the potential problems of intolerance and malabsorption in animals that suffer allergies or intolerances to common fibre sources. The product may also be classed as a natural, whole food; meaning it does not have some of the problems associated with many pharmaceutical treatments, including some negative side effects.

When incorporated in an existing food product format, such as in the examples above, the fibre product described above potentially provides the following benefits to the overall food product:
Tends to increase the fibre content of the food;
May acts as a bulking agent, increasing yield;
Adds moisture to drier formulations (as is commonly the case with gluten-free recipes);
Is allergen-free; and
Tends to reduce the Glycaemic Index (GI) of the food.

In an alternative form, as per example 4 above, the fibre product can be sold as a stand-alone additive that can be used in conjunction with the animals' normal diet, which allows the following advantages:
Can be sold in single or multiple serve packaging for convenience of use;
May be prepared as a drink pudding, or individual sachet;
The dosage can be varied as required for animals with particularly sensitive digestive systems;
May be added to the animals normal meals to aid digestion;
May be a combined with other natural products to enhance the digestive health properties of the animal's diet; and
Tends to reduce the GI of any food with which it is combined.

The supplement tends to provide several benefits to the animal, including increasing the fibre content of the food, having a positive effect on digestion, prolonged control of blood glucose levels and lower blood lipid levels.

It will be appreciated by persons skilled in the art that the above described embodiments are not the only ways in which the invention can be put into practice. There are other alternative embodiments which, while different in some details, nevertheless fall within the scope of the invention, including diets for other types of pet animals.

1. A method of making an animal food product formulated to ameliorate effects of diabetic conditions, comprising:
extracting dietary fibre material from sugar cane and incorporating said fiber material in said food product.

2. The method of claim 1, wherein the sugar cane fibre is prepared via a process including the steps of: subjecting the sugar cane material to at least one wet diffusion step to separate sugars from a residual fibre material; and subjecting the residual fibre material to a rapid, low-heat drying process thereby to enhance the water retention properties of said residual fibre product.

3. The method of claim 2, wherein the pH of the extraction liquid is held between 6.5 and 7.5.

4. The method of claim 3, wherein the extraction step is done under relatively low-shear conditions.

5. The method of claim 2, wherein the wet extraction step is carried out at a temperature in the range 25° C. to 70° C.

6. The method of claim 2, wherein the sugar cane fibre material has undergone a pressure heating step at a pressure in the range 100 psi to 140 psi.

7. A food product formulated to ameliorate the effects of companion animal diabetes; said food product containing dietary fibre material extracted from sugar cane.

8. The food product of claim 7, wherein the sugar cane fibre is prepared via a process including the steps of: subjecting the sugar cane material to at least one wet diffusion step to separate sugars from a residual fibre material; and subjecting the residual fibre material to a rapid, low-heat drying process thereby to enhance the water retention properties of said residual fibre product.

9. The food product of claim 7, wherein, said food product is in the form of a dry powder that can be added to other food products.

10. The food product of claim 7, wherein, said food product is in the form of granules.

11. Treatment of the effects of companion animal diabetes in a companion animal by feeding to said animal a food product incorporating dietary fibre material extracted from sugar cane.

12. The method of treatment of claim 11, wherein the food products is made by preparing sugar cane fibre in a process including the steps of: subjecting the sugar cane material to at least one wet diffusion step to separate sugars from a residual fibre material; and subjecting the residual fibre material to a rapid, low-heat drying process thereby to enhance the water retention properties of said residual fibre product.

13. The food product of claim 8, wherein the sugar cane fibre is prepared via a process wherein the pH of the extraction liquid is held between 6.5 and 7.5.

14. The food product of claim 13, wherein the extraction step is done under relatively low-shear conditions.

15. The food product of claim 8, wherein the wet extraction step is carried out at a temperature in the range 25° C. to 70° C.

16. The food product of claim 8, wherein the sugar cane fibre material has undergone a pressure heating step at a pressure in the range 100 psi to 140 psi.

17. The food product of claim 8, wherein, said food product is in the form of a dry powder that can be added to other food products.

18. The food product of claim 8, wherein, said food product is in the form of granules.

19. The method of treatment of claim 11, wherein the food product is in the form of a dry powder that can be added to other food products.

20. The method of treatment of claim 11, wherein the food product is in the form of granules.
